(19) United States
(12) Patent Application Publication
(10) Pub. No.: US 2005/0256324 A1
(43) Pub. Date: Nov. 17, 2005

(54) Synthesis of amino acid keto-epoxides

(75) Inventors: Guy J. Laidig, Menlo Park, CA (US); Peggy A. Radel, Berkeley, CA (US); Mark S. Smyth, Foster City, CA (US)

Correspondence Address: FISH & NEAVE IP GROUP;ROPES & GRAY LLP, ONE INTERNATIONAL PLACE, BOSTON, MA, 02110-2624, US

(73) Assignee: Proteolix, Inc., South San Francisco, CA (US)

(21) Appl. No.: 11/131,688

(22) Filed: May 17, 2005

Related U.S. Application Data
(63) Continuation-in-part of application No. PCT/US05/16335, filed on May 9, 2005.
(60) Provisional application No. 60/572,072, filed on May 17, 2004.
(60) Provisional application No. 60/569,885, filed on May 10, 2004.
(60) Provisional application No. 60/610,040, filed on Sep. 14, 2004.

(51) Int. Cl. C07D 301/03

(52) U.S. Cl. 549/523; 564/152

(57) ABSTRACT

This invention relates to methods for the preparation of amino acid keto-epoxides. Specifically, allylic ketones are stereoselectively converted to the desired keto epoxides.

### CROSS REFERENCE TO RELATED APPLICATIONS

This application claims the benefit of U.S. Provisional Patent Application Ser. No. 60/572072 filed on May 17, 2004 and is a continuation-in-part of International Application Ser. No. ______ (entitled “Compounds for Enzyme Inhibition”) filed on May 9, 2005. International Application Serial No. ______ (entitled “Compounds for Enzyme Inhibition”) filed on May 9, 2005 claims the benefit of U.S. Provisional Patent Application Ser. No. 60/569885 filed on May 10, 2004 and U.S. Provisional Patent Application Ser. No. 60/610040 filed on Sep. 14, 2004. The teachings of all of the referenced applications are incorporated by reference in their entirety.

### BACKGROUND OF THE INVENTION

A number of compounds that are generally useful as inhibitors of enzymes having a nucleophilic group at the N-terminus have been identified that are structurally related to epoxomicin (Hanada, M., et al. (1992) J. Antibiotics, 45(11): 1746-1752). These compounds are described in U.S. patent application Ser. Nos. 09/569748, 60/562340, 11/106,879, and the PCT filed on May 9, 2005 and are hereby incorporated by reference in their entirety.

There remains a need for an improved process for the production of these compounds in an efficient manner.

### SUMMARY OF THE INVENTION

This invention relates to methods for the synthesis of amino acid keto-epoxides according to scheme (I) 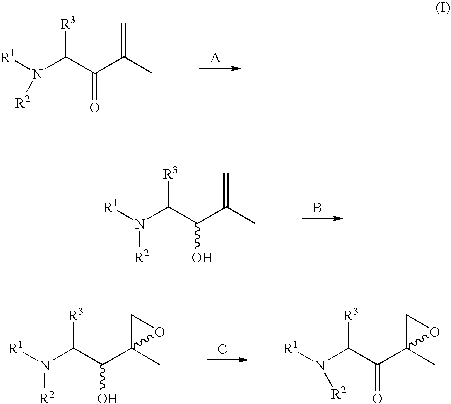
wherein
R¹ is selected from a protecting group or a further chain of amino acids, which itself may be optionally substituted, preferably a protecting group, most preferably an electron withdrawing protecting group;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, heterocyclyl, aryl, heteroaryl, C₁₋₆heteroaralkyl, and C₁₋₆aralkyl;
A is a stereoselective reduction under reducing conditions, preferably sodium borohydride with cerium trichloride, lithium tri-tert-butoxyaluminum hydride, or L-selectride, most preferably sodium borohydride with cerium trichloride;
B is a stereoselective epoxidation under epoxidizing conditions, preferably m-chloroperbenzoic acid or VO(acac)₂ with t-BuOOH, most preferably VO(acac)₂ with t-BuOOH; and
C is an oxidation under oxidizing conditions, preferably Dess-Martin periodinane, or the like, Swem, or tetrapropylammonium perruthenate with 4-methylmorpholine-N-oxide.

Other features and advantages of the invention will be apparent from the following detailed description, and from the claims.

### DETAILED DESCRIPTION OF THE INVENTION

A number of compounds that are generally useful as inhibitors of enzymes having a nucleophilic group at the N-terminus have been identified. The invention describes efficient methods for the production of these compounds. More specifically, described herein are methods for the stereoselective synthesis of amino acid keto-epoxides.

These keto-epoxides may optionally include groups bonded to α′ carbons, the stereochemistry of the α′-carbon (that carbon forming a part of the epoxide or aziridine ring) can be (R) or (S). Note that a preferred compound may have a number of stereocenters having the indicated up-down (or ⊖-α, where β as drawn herein is above the plane of the page) or (R)—(S) relationship (that is, it is not required that every stereocenter in the compound conform to the preferences stated). In some preferred embodiments, the stereochemistry of the α′ carbon is (R), that is, the X atom is β, or above the plane of the molecule. Regarding the stereochemistry, the Cahn-Ingold-Prelog rules for determining absolute stereochemistry are followed. These rules are described, for example, in Organic Chemistry, Fox and Whitesell; Jones and Bartlett Publishers, Boston, Mass. (1994); Section 5-6, pp 177-178, which section is hereby incorporated by reference.

This invention relates to methods for the synthesis of amino acid keto-epoxides according to scheme (I) 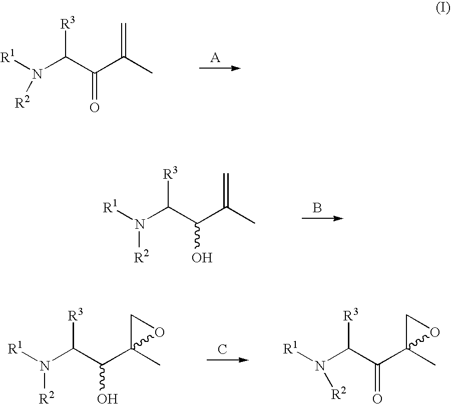
wherein
R¹ is selected from a protecting group or a further chain of amino acids, which itself may be optionally substituted, preferably a protecting group, most preferably an electron withdrawing protecting group;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, heterocyclyl, aryl, heteroaryl, C₁₋₆heteroaralkyl, and C₁₋₆aralkyl;
A is a stereoselective reduction under reducing conditions, preferably wherein the reducing agent is sodium borohydride with cerium trichloride, lithium tri-tert-butoxyaluminum hydride, or L-selectride, most preferably sodium borohydride with cerium trichloride;
B is a stereoselective epoxidation under epoxidizing conditions, preferably wherein the oxidizing reagent(s) is m-chloroperbenzoic acid or VO(acac)₂ with t-BuOOH, most preferably VO(acac)₂ with t-BuOOH; and
C is an oxidation under oxidizing conditions, preferably a Swern oxidation or an oxidation wherein the oxidizing reagent(s) is Dess-Martin periodinane, or the like, or tetrapropylammonium perruthenate (TPAP) with 4-methylmorpholine-N-oxide (NMO), most preferably a Swern oxidation.

The use of various N-protecting groups, e.g., the benzyloxy carbonyl group or the t-butyloxycarbonyl group (Boc), various coupling reagents, e.g., dicyclohexylcarbodiimide (DCC), 1,3-diisopropylcarbodiimide (DIC), 1-(3-dimethylaminopropyl)-3-ethylcarbodiimide (EDC), N-hydroxyazabenzotriazole (HATU), carbonyldiimidazole, or 1-hydroxybenzotriazole monohydrate (HOBT), and various cleavage conditions: for example, trifluoracetic acid (TFA), HCl in dioxane, hydrogenation on Pd-C in organic solvents (such as methanol or ethyl acetate), boron tris(trifluoroacetate), and cyanogen bromide, and reaction in solution with isolation and purification of intermediates are well-known in the art of peptide synthesis, and are equally applicable to the preparation of the subject compounds (Greene, T. W.; Wuts, P. G. M. Protective Groups in Organic Synthesis, 3^{rd} ed.; Wiley: N.Y., 1999).

In certain embodiments, R¹ is selected from a protecting group or a further chain of amino acids, which itself may be optionally substituted. In preferred embodiments, R¹ is a protecting group. In a more preferred embodiment, R¹ is an electron withdrawing protecting group. In certain such embodiments, R¹ is selected from t-butoxy carbonyl (Boc), benzoyl (Bz), fluoren-9-ylmethoxycarbonyl (Fmoc), trichloroethoxycarbonyl (Troc), and benzyloxy carbonyl (Cbz). In the most preferred embodiment, R¹ is Cbz.

In certain embodiments, R¹ and R² together are C(O)-aryl-C(O) or C(O)C₁₋₆alkenylC(O), thereby forming a ring. In a preferred such embodiment, R¹ and R² together are phthaloyl.

In certain embodiments, R³ is selected from hydrogen, C₁₋₆alkyl, C₁₋₆alkoxyalkyl, heterocyclyl, aryl, heteroaryl, C₁₋₆heteroaralkyl, and C₁₋₆aralkyl. In preferred embodiments, R³ is C₁₋₆alkyl. In the most preferred embodiment, R³ is isobutyl.

A is a stereoselective reduction under reducing conditions. In a preferred embodiment, the reducing agent in A is selected from sodium borohydride with cerium trichloride, lithium tri-tert-butoxyaluminum hydride, or L-selectride. In a more preferred embodiment, the reducing agent is sodium borohydride with cerium trichloride. Other suitable agents include, but are not limited to achiral reducing agents such as lithium aluminum hydride, trimethoxylithium aluminum hydride, K-selectride, KS-selectride, LS-selectride, and diisobutylaluminum hydride, chiral reducing agents such as (R) or (S)-2-methyl-CBS-oxaborolidine and (R) or (S)-alpine borane, chiral oxazaborolidines, (R,R or S,S) lithium dimethylborolane, and chiral alkoxy(acyloxy)borohydrides, or achiral reducing agents in the presence of chiral additives such as lithium aluminum hydride in the presence of quinine or ephedrine.

B is a stereoselective epoxidation under epoxidizing conditions. In preferred embodiments, the conditions include m-chloroperbenzoic acid (or another suitable peroxyacid) or VO(acac)₂ with t-BuOOH. In the most preferred embodiment B is VO(acac)₂ with t-BuOOH. Other suitable epoxidizing conditions include, but are not limited to, Sharpless asymmetric epoxidation, Shi asymmetric epoxidation, Jacobsen epoxidation, dimethyldioxirane, and trifluoromethylmethyldioxirane.

C is an oxidation under oxidizing conditions. In preferred embodiments, C is a Swern or Moffat oxidation or employs Dess-Martin periodinane or TPAP with NMO. In the most preferred embodiment, C is a Swern oxidation. Other suitable oxidizing agents include, but are not limited to, ruthenium dioxide, pyridinium chlorochromate (PCC), IBX, and pyridinium dichromate (PDC).

In certain embodiments, the invention provides the sequence of reactions as discrete steps, wherein the product of each reaction (A, B, and C) is isolated and purified. In another embodiment, the invention provides the sequence of reactions, wherein the product of at least one reaction is used in the next reaction without isolation and/or purification. Additionally, the invention relates to the sequence of reactions (A, B, and C), each individual reaction (A, B, and C) and subcombinations thereof. Thus, the invention relates to the synthesis of an allyl alcohol according to scheme (II) 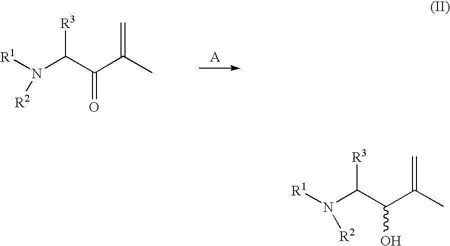
wherein
R¹ is selected from a protecting group or a further chain of amino acids, which itself may be optionally substituted, preferably a protecting group, most preferably an electron withdrawing protecting group;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, heterocyclyl, aryl, heteroaryl, C₁₋₆heteroaralkyl, and C₁₋₆aralkyl; and
A is a stereoselective reduction under reducing conditions, preferably wherein the reducing agent is sodium borohydride with cerium trichloride, lithium tri-tert-butoxyaluminum hydride, or L-selectride, most preferably sodium borohydride with cerium trichloride.

The invention further relates to the synthesis of an epoxide according to scheme (III) 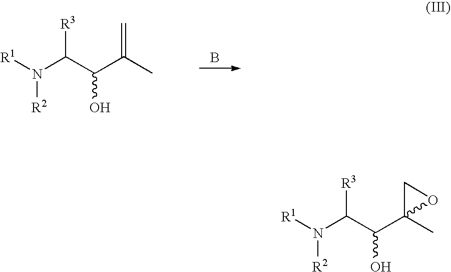
wherein
R¹ is selected from a protecting group or a further chain of amino acids, which itself may be optionally substituted, preferably a protecting group, most preferably an electron withdrawing protecting group;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, heterocyclyl, aryl, heteroaryl, C₁₋₆heteroaralkyl, and C₁₋₆aralkyl; and
B is a stereoselective epoxidation under epoxidizing conditions, preferably wherein the oxidizing reagent(s) is m-chloroperbenzoic acid or VO(acac)₂ with t-BuOOH, most preferably VO(acac)₂ with t-BuOOH.

Additionally, the invention relates to the synthesis of arino acid keto-epoxides according to scheme (IV) 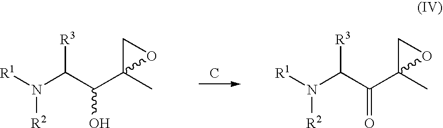
wherein
R¹ is selected from a protecting group or a further chain of amino acids, which itself may be optionally substituted, preferably a protecting group, most preferably an electron withdrawing protecting group;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, heterocyclyl, aryl, heteroaryl, C₁₋₆heteroaralkyl, and C₁₋₆aralkyl; and
C is an oxidation under oxidizing conditions, preferably a Swern oxidation or an oxidation wherein the oxidizing reagent(s) is Dess-Martin periodinane or tetrapropylammonium perruthenate (TPAP) with 4-methylmorpholine-N-oxide (NMO), most preferably a Swern oxidation.

In another embodiment, the reduction may be replaced by an organometal addition to an aldehyde, followed by an epoxidation oxidation sequence as shown in scheme (V)

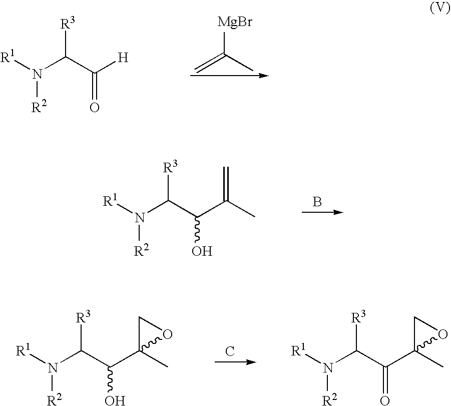

Alternatively, the epoxidation could be replaced with an asymmetric dihydroxylation, selective silylation or tosylation, epoxidation sequence as shown in scheme (VI) to arrive at the desired amino acid keto-epoxide.

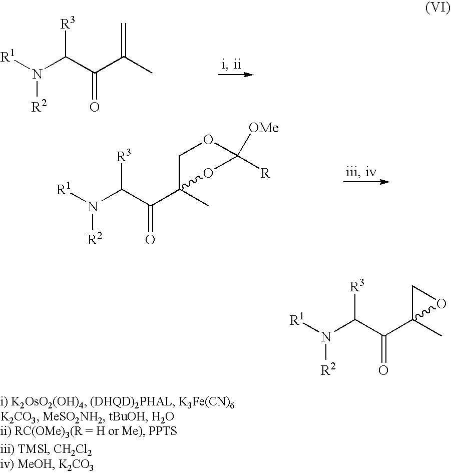

In some embodiments, the amino acid keto-epoxide may optionally be further modified in a four-step procedure (Wipf, P. et al., 1998, J. Org. Chem., 63:6089-6090) or a 5 one-step procedure (Shao, H. et al., 1995, J. Org. Chem., 60:790-791) resulting in the formation of the corresponding aziridine.

In certain embodiments, the compounds in scheme I have the following stereochemistry

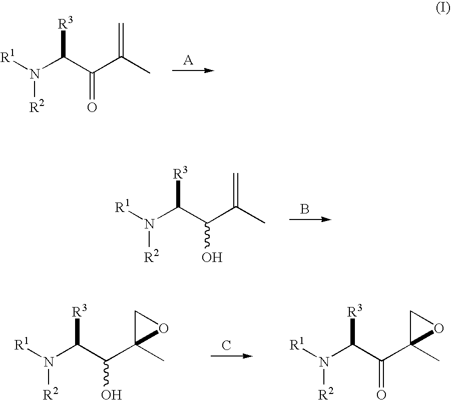

In certain embodiments, the amino acid keto-epoxide or keto-aziridine may be further modified by deprotection of the amine, if applicable, and coupling with a chain of amino acids. Methods for the coupling of such fragments are well known in the art (Elofsson, M., et al. (1999) Chemistry& Biology, 6:811-822; Elofsson, M., et al (1999) Chemistry& Biology, 6:811-822). In a preferred embodiment, the chain of amino acids comprises one to three amino acids.

In certain embodiments, the chain of amino acids has a structure of formula (I) or a pharmaceutically acceptable salt thereof 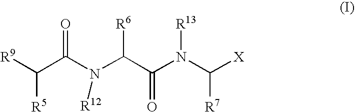
wherein each A is independently selected from C═O, C═S, and SO₂, preferably C═O; or
A is optionally a covalent bond when adjacent to an occurrence of Z;
L is absent or is selected from C═O, C═S, and SO₂, preferably L is absent or C═O;
M is absent or is C₁₋₂alkyl, preferably C₁₋₈alkyl;
Q is absent or is selected from O, NH, and N—C₁₋₆alkyl, preferably Q is absent, O, or NH, most preferably Q is absent or O;
X is COOH or an activated form thereof, preferably X is COOH, COCl, or CON(Me)(OMe), most preferably X is COOH or COCl;
Y is absent or is selected from O, NH, N—C₁₋₆alkyl, S, SO, SO₂, CHOR¹⁷, and CHCO₂R¹⁷; each Z is independently selected from O, S, NH, and N—C₁₋₆-alkyl, preferably O; or
Z is optionally a covalent bond when adjacent to an occurrence of A;
R⁵, R⁶, and R⁷ are each independently selected from C₁₋₆alkyl, C₁₋₆ydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₆alkyl and C₁₋₅alkyl ester and aryl ester), thiol, or thioether substituents;
R⁹ is N(R¹⁰)LQR¹¹;
R¹⁰, R¹², and R¹³ are independently selected from hydrogen, OH, C₁₋₆alkyl, and a group of formula II; preferably, R¹⁰ is selected from hydrogen, OH, and C₁₋₆alkyl, and R¹² and R¹³ are independently selected from hydrogen and C₁₋₆alkyl, preferably hydrogen; 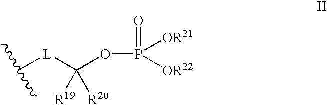
R¹¹ is selected from hydrogen, C₁₋₆alkyl, C₁₋₆alkenyl, C₁₋₆alkynyl, aryl, C₁₋₆aralkyl, heteroaryl, C₁₋₆heteroaralkyl, R¹⁵ZAZ-C₁₋₈alkyl-, R¹⁸Z-C₁₋₈alkyl-, (R¹⁵O)(R¹⁶O)P(═O)O—C₁₋₈alkyl-ZAZ-C₁₋₈alkyl-, R¹⁵ZAZ-C₁₋₈alkyl-ZAZ-C₁₋₈alkyl-, heterocyclylMZAZ-C₁₋₈alkyl-, (R¹⁵O)(R¹⁶O)P(═O)O—C₁₋₈alkyl-, (R¹⁷)₂N—C₁₋₁₂alkyl-, (R¹⁷)₃N⁺—C₁₋₂alkyl-, heterocyclylM-, carbocyclylM-, R¹⁸SO₂C₁₋₈alkyl-, and R¹⁸SO₂NH; preferably C₁₋₆alkyl, C₁₋₆alkenyl, C₁₋₆alkynyl, aryl, C₁₋₆aralkyl, heteroaryl, C₁₋₆heteroaralkyl, R¹⁵ZA—C₁₋₈alkyl-, R¹⁸Z—C₁₋₈alkyl-, (R¹⁵O)(R¹⁶O)P(═O)O—C₁₋₈alkyl-ZAZ-C₁₋₈alkyl-, (R¹⁵O)(R¹⁶O)P(═O)O—C₁₋₈alkyl-Z-C₁₋₈alkyl-, R¹⁵ZA-C₁₋₈alkyl-ZAZ-C₁₋₈alkyl-, heterocyclytMZAZ-C₁₋₈alkyl-, (R¹⁵O)(R¹⁶O)P(═O)O—C₁₋₈alkyl-, (R¹⁷)₂N—C₁₋₈alkyl-, (R¹⁷)₃N⁺—C₁₋₈alkyl-, heterocyclylM-, carbocyclylM-, R¹⁸SO₂C₁₋₈alkyl-, and R¹⁸SO₂NH, wherein each occurrence of Z and A is independently other than a covalent bond; or
R¹⁰ and R¹¹ together are C₁₋₆alkyl-Y—C₁₋₆alkyl, C₁₋₆alkyl-ZAZ-C₁₋₆alkyl, ZAZ-C₁₋₆alkyl-ZAZ-C₁₋₆alkyl, ZAZ-C₁₋₆alkyl-ZAZ, or C₁₋₆alkyl-A, thereby forming a ring; preferably C₁₋₂alkyl-Y—C₁₋₂alkyl, C₁₋₂alkyl-ZA-C₁₋₂alkyl, A—C₁₋₂alkyl-ZA-C₁₋₂alkyl, A-C₁₋₃alkyl-A, or C₁₋₄alkyl-A, wherein each occurrence of Z and A is independently other than a covalent bond;
R¹⁵ and R¹⁶ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R¹⁵ and R¹⁶ together are C₁₋₆alkyl, thereby forming a ring;
each R¹⁷ is independently selected from hydrogen and C₁₋₆alkyl, preferably C₁₋₆alkyl;
R¹⁸ is independently selected from hydrogen, OH, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl;
R¹⁹ and R²⁰ are independently selected from hydrogen and C₁₋₆alkyl, or R¹⁹ and R²⁰ together form a 3- to 6-membered carbocyclic or heterocyclic ring; and
R²¹ and R²² are independently selected from hydrogen, a metal cation, C₁₋₆alkyl, and C₁₋₆aralkyl, or R²¹ and R²² together represent C₁₋₆alkyl, thereby forming a ring;
provided that in any occurrence of the sequence ZAZ, at least one member of the sequence must be other than a covalent bond.

In some embodiments, R⁵, R⁶, and R⁷ are selected from C₁₋₆alkyl or C₁₋₆aralkyl. In preferred embodiments, R⁶ is C₁₋₆alkyl and R⁵ and R⁷ are C₁₋₆aralkyl. In the most preferred embodiment, R⁶ is isobutyl, R⁵ is 2-phenylethyl, and R⁷ is phenylmethyl.

In certain embodiments, L and Q are absent and R¹¹ is selected from C₁₋₆alkyl, C₁₋₆alkenyl, C₁₋₆-alkynyl, C₁₋₆aralkyl, and C₁₋₆heteroaralkyl. In certain such embodiments, R¹⁰ is C₁₋₆alkyl and R¹¹ is selected from butyl, allyl, propargyl, phenylmethyl, 2-pyridyl, 3-pyridyl, and 4-pyridyl.

In other embodiments, L is SO₂, Q is absent, and R¹¹ is selected from C₁₋₆alkyl and aryl. In certain such embodiments, R¹¹ is selected from methyl and phenyl.

In certain embodiments, L is C═O and R¹¹ is selected from C₁₋₆alkyl, C₁₋₆-alkenyl, C₁₋₆alkynyl, aryl, C₁₋₆aralkyl, heteroaryl, C₁₋₆heteroaralkyl, R¹⁵ZA-C₁₋₈alkyl-, R¹⁸Z-C₁₋₈alkyl-, (R¹⁵O)(R¹⁶O)P(═O)O—C₁₋₈alkyl-, (R¹⁵O)(R¹⁶O)P(═O)O—C₁₋₈alkyl-ZAZ-C₁₋₈alkyl-, (R¹⁵O)(R¹⁶O)P(═O)O—C₁₋₁₈alkyl-Z-C₁₋₈alkyl-, R¹⁵ZA-C₁₋₈alkyl-ZAZ-C₁₋₈alkyl-, heterocyclylMZAZ-C₁₋₈alkyl-, (R¹⁷)₂N—C₁₋₈alkyl-, (R¹⁷)₃N⁺—C₁₋₈alkyl-, heterocyclylM-, carbocyclylM-, R¹⁸SO₂C₁₋₈alkyl-, and R¹⁸SO₂NH—, wherein each occurrence of Z and A is independently other than a covalent bond. In certain embodiments, L is C═O, Q is absent, and R¹¹ is H.

In certain embodiments, R¹⁰ is C₁₋₆alkyl, R¹¹ is C₁₋₆alkyl, Q is absent, and L is C═O. In certain such embodiments, R¹¹ is ethyl, isopropyl, 2,2,2-trifluoroethyl, or 2-(methylsulfonyl)ethyl.

In other embodiments, L is C═O, Q is absent, and R¹¹ is C₁₋₆aralkyl. In certain such embodiments, R¹¹ is selected from 2-phenylethyl, phenylmethyl, (4-methoxyphenyl)methyl, (4-chlorophenyl)methyl, and (4-fluorophenyl)methyl.

In other embodiments, L is C═O, Q is absent, R¹⁰ is C₁₋₆alkyl, and R¹¹ is aryl. In certain such embodiments, R¹¹ is substituted or unsubstituted phenyl.

In certain embodiments, L is C═O, Q is absent or O, n is 0 or 1, and R¹¹ is —(CH₂)ₙcarbocyclyl. In certain such embodiments, R¹¹ is cyclopropyl or cyclohexyl.

In certain embodiments, L and A are C═O, Q is absent, Z is O, n is an integer from 1 to 8 (preferably 1), and R¹¹ is selected from R¹⁵ZA-C₁₋₈alkyl-, R¹⁸Z-C₁₋₈alkyl-, R¹⁵ZA-C₁₋₈alkyl-ZAZ-C₁₋₈alkyl-, (R¹⁵O)(R¹⁶O)P(═O)O—C₁₋₈alkyl-ZAZ-C₁₋₈alkyl-, (R¹⁵O)(R¹⁶O)P(═O)O—C₁₋₈alkyl-Z-C₁₋₈alkyl-, and heterocyclylMZAZ-C₁₋₈alkyl-, wherein each occurrence of A is independently other than a covalent bond. In certain such embodiments, R⁷ is heterocyclylMZAZ-C₁₋₈alkyl- where heterocyclyl is substituted or unsubstituted oxodioxolenyl or N(R¹²)(R¹³), wherein R¹² and R¹³ together are C₁₋₆alkyl-Y—C₁₋₆alkyl, preferably C₁₋₃alkyl-Y—C₁₋₃alkyl, thereby forming a ring.

In certain preferred embodiments, L is C═O, Q is absent, n is an integer from 1 to 8, and R¹¹ is selected from (R¹⁵O)(R¹⁶O)P(═O)O—C₁₋₈alkyl-, (R¹⁷)₂NC₁₋₈alkyl, (R¹⁷)₃N⁺(CH₂)ₙ—, and heterocyclyl-M-. In certain such embodiments, R¹¹ is —C₁₋₈alkylN(R¹⁷)₂ or —C₁₋₈alkylN⁺(R¹⁷)₃, where R¹⁷ is C₁₋₆alkyl. In certain other such embodiments, R¹¹ is heterocyclylM-, where heterocyclyl is selected from morpholino, piperidino, piperazino, and pyrrolidino.

In certain embodiments, L is C═O, R¹⁰ is C₁₋₆alkyl, Q is selected from O and NH and R¹¹ is selected from C₁₋₆alkyl, cycloalkyl-M, C₁₋₆aralkyl, and C₁₋₆heteroaralkyl. In other embodiments, L is C═O, R¹⁰ is C₁₋₆alkyl, Q is selected from O and NH, and R¹¹ is C₁₋₆alkyl, where C₁₋₆alkyl is selected from methyl, ethyl, and isopropyl. In further embodiments, L is C═O, R¹⁰ is C₁₋₆alkyl, Q is selected from O and NH and R¹¹ is C₁₋₆aralkyl, where aralkyl is phenylmethyl. In other embodiments, L is C═O, R¹⁰ is C₁₋₆alkyl, Q is selected from O and NH, and R¹¹ is C₁₋₆heteroaralkyl, where heteroaralkyl is (4-pyridyl)methyl.

In certain embodiments, L is absent or is C═O, and R¹⁰ and R¹¹ together are C₁₋₆alkyl-Y—C₁₋₆alkyl, C₁₋₆alkyl-ZA-C₁₋₆alkyl, or C₁₋₆alkyl-A, wherein each occurrence of Z and A is independently other than a covalent bond, thereby forming a ring. In certain preferred embodiments, L is C═O, Q and Y are absent, and R¹⁰ and R¹¹ together are C₁₋₃alkyl-Y—C₁₋₃alkyl. In another preferred embodiment, L and Q are absent, and R¹⁰ and R¹¹ together are C₁₋₃alkyl-Y—C₁₋₃alkyl. In another preferred embodiment, L is C═O, Q is absent, Y is selected from NH and N—C₁₋₆alkyl, and R¹⁰ and R¹¹ together are C₁₋₃alkyl-Y—C₁₋₃alkyl. In another preferred embodiment, L is C═O, Y is absent, and R¹⁰ and R¹¹ together are C₁₋₃alkyl-Y—C₁₋₃alkyl. In another preferred embodiment, L and A are C═O, and R¹⁰ and R¹¹ together are C₁₋₂alkyl-ZA-C₁₋₂alkyl. In another preferred embodiment, L and A are C═O and R¹⁰ and R¹¹ together are C₂₋₃alkyl-A.

In certain embodiments, the chain of amino acids has a structure of formula (III) 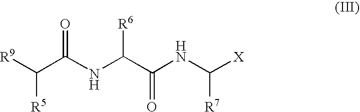
wherein
each A is independently selected from C═O, C═S, and SO₂, preferably C═O; or
A is optionally a covalent bond when adjacent to an occurrence of Z;
each B is independently selected from C═O, C═S, and SO₂, preferably C═O;
D is absent or is C₁₋₈alkyl;
G is selected from O, NH, and N—C₁₋₆alkyl;
K is absent or is selected from C═O, C═S, and SO₂, preferably K is absent or is C═O;
L is absent or is selected from C═O, C═S, and SO₂, preferably L is absent or C═O;
M is absent or is C₁₋₈alkyl;
Q is absent or is selected from O, NH, and N—C₁₋₆alkyl, preferably Q is absent, O, or NH, most preferably Q is absent;
X is COOH or an activated form thereof, preferably X is COOH, COCl, or CON(Me)(OMe), most preferably X is COOH or COCl;
each V is independently absent or is selected from O, S, NH, and N—C₁₋₆alkyl, preferably V is absent or O;
W is absent or is independently selected from O, S, NH, and N—C₁₋₆alkyl, preferably O;
Y is absent or is selected from O, NH, N—C₁₋₆alkyl, S, SO, SO₂, CHOR¹⁷, and CHCO₂R¹⁷;
each Z is independently selected from O, S, NH, and N—C₁₋₆alkyl, preferably O; or
Z is optionally a covalent bond when adjacent to an occurrence of A;
R⁵, R⁶, and R⁷ are each independently selected from C₁₋₆alkyl, C₁₋₆hydroxyalkyl, C₁₋₆alkoxyalkyl, aryl, C₁₋₆aralkyl, and R¹⁶DVKOC₁₋₃alkyl-, wherein at least one of R⁵ and R⁷ is R¹⁶DVKOC₁₋₃alkyl-;
R⁹ is N(R¹⁰)LQR¹¹;
R¹⁰ is selected from hydrogen, OH, and C₁₋₆alkyl, preferably hydrogen or C₁₋₆alkyl;
R¹¹ is a further chain of amino acids, hydrogen, a protecting group, aryl, or heteroaryl, any of which is optionally substituted with halogen, carbonyl, nitro, hydroxy, aryl, C₁₋₅alkyl; or R¹¹ is selected from C₁₋₆alkyl, C₁₋₆alkenyl, C₁₋₆alkynyl, C₁₋₆aralkyl, C₁₋₆heteroaralkyl, R¹²ZAZ-C₁₋₈alkyl-, R¹⁵ZAZ-C₁₋₈alkyl-, (R¹²O)(R¹³O)P(═O)O—C₁₋₈alkyl-ZAZ-C₁₋₈alkyl-, R¹²ZAZ-C₁₋₈alkyl-ZAZ-C₁₋₈alkyl-, heterocyclylMZAZ-C₁₋₈alkyl-, (R¹²O)(R¹³O)P(═O)O—C₁₋₈alkyl-, (R¹⁴)₂N—C₁₋₈alkyl-, (R¹⁴)₃N⁺—C₁₋₈alkyl-, heterocyclylM-, carbocyclylM-, R¹⁵SO₂C₁₋₈alkyl-, and R¹⁵SO₂NH; or
R¹⁰ and R¹¹ together are C₁₋₆alkyl-Y—C₁₋₆alkyl, C₁₋₆alkyl-ZAZ-C₁₋₆alkyl, ZAZ-C₁₋₆alkyl-ZAZ-C₁₋₆alkyl, ZAZ-C₁₋₆alkyl-ZAZ, or C₁₋₆alkyl-ZAZ;
R¹² and R¹³ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R¹² and R¹³ together are C₁₋₆alkyl, thereby forming a ring;
each R¹⁴ is independently selected from hydrogen and C₁₋₆alkyl, preferably C₁₋₆alkyl;
each R¹⁵ is independently selected from hydrogen, OR¹⁴, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl;
R¹⁶ is selected from hydrogen, (R¹⁷O)(R¹⁸O)P(═O)W—, R¹⁷GB-, heterocyclyl-, (R¹⁹)₂N—, (R¹⁹)₃N⁺—, R¹⁹SO₂GBG-, and R¹⁷GBC₁₋₈alkyl- where the C₁₋₈alkyl moiety is optionally substituted with OH, C₁₋₈alkylW (optionally substituted with halogen, preferably fluorine), aryl, heteroaryl, carbocyclyl, heterocyclyl, and C₁₋₆aralkyl, preferably at least one occurrence of R¹⁶ is other than hydrogen;
R¹⁷ and R¹⁸ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R¹⁷ and R¹⁸ together are C₁₋₆alkyl, thereby forming a ring; and
each R¹⁹ is independently selected from hydrogen, OR¹⁴, C₁₋₆alkyl, C₁₋₆-alkenyl, C₁₋₆akynyl, carbocyclyl, heterocyclyl, aryl, heteroaryl, C₁₋₆aralkyl, and C₁₋₆heteroaralkyl; and
D, G, V, K, and W are selected such that there are no O—O, N—O, S—N, or S—O bonds.

In certain embodiments, R⁵, R⁶, and R⁷ are each independently selected from C₁₋₆alkyl, C₁₋₆hydroxyalkyl, C₁₋₆alkoxyalkyl, aryl, C₁₋₆aralkyl, and R¹⁶DVKOC₁₋₃alkyl- wherein at least one of R⁵ and R⁷ is R¹⁶DVKOC₁₋₃alkyl-. In preferred embodiments, one of R⁵ and R⁷ is C₁₋₆aralkyl and the other is R¹⁶DVKOC₁₋₃alkyl-, and R⁶ is independently C₁₋₆alkyl. In the most preferred embodiment, one of R⁵ and R⁷ is 2-phenylethyl or phenylmethyl and the other is R¹⁶DVKOCH₂- or R¹⁶DVKO(CH₃)CH—, and R⁶ is isobutyl.

In certain embodiments, each R¹⁵ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl.

In certain embodiments, each R¹⁹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl.

In certain embodiments, L and Q are absent and R¹¹ is selected from hydrogen, a further chain of amino acids, C₁₋₆acyl, a protecting group, aryl, heteroaryl, C₁₋₆alkyl, C₁₋₆alkenyl, C₁₋₆alkynyl, C₁₋₆aralkyl, and C₁₋₆heteroaralkyl. In certain such embodiments, R¹⁰ is C₁₋₆alkyl and R¹¹ is selected from butyl, allyl, propargyl, phenylmethyl, 2-pyridyl, 3-pyridyl, and 4-pyridyl.

In other embodiments, L is SO₂, Q is absent, and R¹¹ is selected from C₁₋₆alkyl and aryl. In certain such embodiments, R¹¹ is selected from methyl and phenyl.

In certain embodiments, L is C═O and R¹¹ is selected from C₁₋₆alkyl, C₁₋₆alkenyl, C₁₋₆alkynyl, aryl, C₁₋₆aralkyl, heteroaryl, C₁₋₆heteroaralkyl, R¹²ZA-C₁₋₈alkyl-, R¹⁵Z-C₁₋₈alkyl-, (R¹²O)(R¹³O)P(═O)O—C₁₋₈alkyl-, (R¹²O)(R¹³O)P(═O)O—C₁₋₈alkyl-ZAZ-C₁₋₈alkyl-, (R¹²O)(R¹³O)P(═O)O—C₁₋₈alkyl-Z-C₁₋₈alkyl-, R¹²ZA-C₁₋₈alkyl-ZAZ-C₁₋₈alkyl-, heterocyclylMZAZ-C₁₋₈alkyl-, (R¹⁴)₂N—C₁₋₈alkyl-, (R¹⁴)₃N⁺—C₁₋₈alkyl-, heterocyclylM-, carbocyclylM-, R¹⁵SO₂C₁₋₈alkyl-, and R¹⁵SO₂NH—. In certain embodiments, L is C═O, Q is absent, and R¹¹ is H.

In certain embodiments, R¹⁰ is C₁₋₆alkyl, R¹¹ is C₁₋₆alkyl, Q is absent, and L is C═O. In certain such embodiments, R¹¹ is ethyl, isopropyl, 2,2,2-trifluoroethyl, or 2-(methylsulfonyl)ethyl.

In other embodiments, L is C═O, Q is absent, and R¹¹ is C₁₋₆aralkyl. In certain such embodiments, R¹¹ is selected from 2-phenylethyl, phenylmethyl, (4-methoxyphenyl)methyl, (4-chlorophenyl)methyl, and (4-fluorophenyl)methyl.

In other embodiments, L is C═O, Q is absent, R¹⁰ is C₁₋₆alkyl, and R¹¹ is aryl. In certain such embodiments, R¹¹ is substituted or unsubstituted phenyl.

In certain embodiments, L is C═O, Q is absent or O, and R¹¹ is —(CH₂)ₙcarbocyclyl. In certain such embodiments, R¹¹ is cyclopropyl or cyclohexyl.

In certain embodiments, L and A are C═O, Q is absent, Z is O, and R¹¹ is selected from R¹²ZA-C₁₋₈alkyl-, R¹⁵Z-C₁₋₈alkyl-, R¹²ZA-C₁₋₈alkyl-ZAZ-C₁₋₈alkyl-, (R¹²O)(R¹³O)P(═O)O—C₁₋₈alkyl-ZAZ-C₁₋₈alkyl-, (R¹²O)(R¹³O)P(═O)O—C₁₋₈alkyl-Z-C₁₋₈alkyl-, and heterocyclylMZAZ-C₁₋₈alkyl-. In certain such embodiments, R¹¹ is heterocyclylMZAZ-C₁₋₈alkyl- where heterocyclyl is substituted or unsubstituted oxodioxolenyl or N(R²⁰)(R²¹), wherein R²⁰ and R²¹ together are C₁₋₆alkyl-Y—C₁₋₆alkyl, preferably C₁₋₃alkyl-Y—C₁₋₃alkyl, thereby forming a ring.

In certain preferred embodiments, L is C═O, Q is absent, and R¹¹ is selected from (R¹²O)(R¹³O)P(═O)O—C₁₋₈alkyl-, (R¹⁴)₂NC₁₋₈alkyl, (R¹⁴)₃N⁺(CH₂)ₙ—, and heterocyclyl-M-. In certain such embodiments, R¹¹ is —C₁₋₈alkylN(R¹⁴)₂ or —C₁₋₈alkylN⁺(R¹⁴)₃, where R¹⁴ is C₁₋₆alkyl. In certain other such embodiments, R¹¹ is heterocyclylM-, where heterocyclyl is selected from morpholino, piperidino, piperazino, and pyrrolidino.

In certain embodiments, L is C═O, R¹⁰ is C₁₋₆alkyl, Q is selected from O and NH and R¹¹ is selected from C₁₋₆alkyl, cycloalkyl-M, C₁₋₆araalkyl, and C₁₋₆heteroaraalkyl. In other embodiments, L is C═O, R¹⁰ is C₁₋₆alkyl, Q is selected from O and NH, and R¹¹ is C₁₋₆alkyl, where C₁₋₆alkyl is selected from methyl, ethyl, and isopropyl. In further embodiments, L is C═O, R¹⁰ is C₁₋₆alkyl, Q is selected from O and NH and R¹¹ is C₁₋₆aralkyl, where aralkyl is phenylmethyl. In other embodiments, L is C═O, R¹⁰ is C₁₋₆alkyl, Q is selected from O and NH, and R¹¹ is C₁₋₆heteroaralkyl, where heteroaralkyl is (4-pyridyl)methyl.

In certain embodiments, L is absent or is C═O, and R¹⁰ and R¹¹ together are C₁₋₆alkyl-Y—C₁₋₆alkyl, C₁₋₆alkyl-ZA-C₁₋₆alkyl, or C₁₋₆alkyl-A, thereby forming a ring. In certain preferred embodiments, L is C═O, Q and Y are absent, and R¹⁰ and R¹¹ together are C₁₋₃alkyl-Y—C₁₋₃alkyl. In another preferred embodiment, L and Q are absent, and R¹⁰ and R¹¹ together are C₁₋₃alkyl-Y—C₁₋₃alkyl. In another preferred embodiment, L is C═O, Q is absent, Y is selected from NH and N—C₁₋₆alkyl, and R¹⁰ and R¹¹ together are C₁₋₃alkyl-Y—C₁₋₃alkyl. In another preferred embodiment, L is C═O, Y is absent, and R¹⁰ and R¹¹ together are C₁₋₃alkyl-Y—C₁₋₃alkyl. In another preferred embodiment, L and A are C═O, and R¹⁰ and R¹¹ together are C₁₋₂alkyl-ZA-C₁₋₂alkyl. In another preferred embodiment, L and A are C═O and R¹⁰ and R¹¹ together are C₂₋₃alkyl-A.

In certain embodiments, R¹⁶ is (R¹⁷O)(R¹⁸O)P(═O)W—. In certain such embodiments, D, V, K, and W are absent. In other such embodiments, V and K are absent, D is C₁₋₈alkyl, and W is O. In yet other such embodiments, D is C₁₋₈alkyl, K is C═O, and V and W are O.

In certain embodiments, R¹⁶ is R¹⁷GB—. In preferred embodiments, B is C═O, G is O, D is C₁₋₈alkyl, V is O, and K is C═O.

In certain embodiments, R¹⁶ is heterocyclyl-. In preferred such embodiments, D is C₁₋₈alkyl. In certain such embodiments, V is O, K is C═O, and heterocyclyl is oxodioxolenyl. In other such embodiments, V is absent, K is absent or is C═O, and heterocyclyl is N(R²⁰)(R²¹), where R²⁰ and R²¹ together are J-T-J, J-WB-J, or B-J-T-J, T is absent or is selected from O, NR¹⁷, S, SO, SO₂, CHOR¹⁹, CHCO₂R¹⁷, C═O, CF₂, and CHF, and J is absent or is C₁₋₃alkyl.

In certain embodiments, R¹⁶ is (R¹⁹)₂N— or (R¹⁹)₃N⁺—, and preferably V is absent. In preferred such embodiments, D is C₁₋₈alkyl and K is absent or C═O. In certain embodiments where V is absent and R¹⁶ is (R¹⁹)₂N—, D is absent K is absent or is C═O, preferably K is C═O.

In certain embodiments, R¹⁶ is R¹⁹SO₂GBG-. In preferred such embodiments, B is C═O, D, V, and K are absent, and G is NH or NC₁₋₆alkyl.

In certain embodiments, R¹⁶ is R¹⁷GBC₁₋₈alkyl-. In preferred embodiments, B is C═O, G is O, and the C₁₋₈alkyl moiety is optionally substituted with OH, C₁₋₈alkyl (optionally substituted with halogen, preferably fluorine), C₁₋₈alkylW, aryl, heteroaryl, carbocyclyl, heterocyclyl, and C₁₋₆aralkyl. In certain such embodiments, the C₁₋₈alkyl moiety is an unsubstituted, mono-, or disubstituted C₁alkyl.

In certain embodiments, the product of the coupling reaction of a compound of the amino acid keto-epoxide or keto-aziridine with a compound of formula (III) is a compound having a structure of formula (IV) 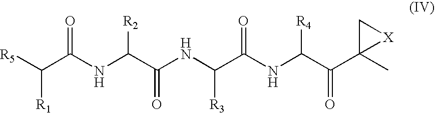
wherein
each A is independently selected from C═O, C═S, and SO₂, preferably C═O; or
A is optionally a covalent bond when adjacent to an occurrence of Z;
each B is independently selected from C═O, C═S, and SO₂, preferably C═O;
D is absent or is C₁₋₈alkyl;
G is selected from O, NH, and N—C₁₋₆alkyl;
K is absent or is selected from C═O, C═S, and SO₂, preferably K is absent or is C═O;
L is absent or is selected from C═O, C═S, and SO₂, preferably L is absent or C═O;
M is absent or is C₁₋₈alkyl;
Q is absent or is selected from O, NH, and N—C₁₋₆alkyl, preferably Q is absent, O, or NH, most preferably Q is absent;
X is selected from O, S, NH, and N—C₁₋₆alkyl, preferably O;
each V is independently absent or is selected from O, S, NH, and N—C₁₋₆alkyl, preferably V is absent or O;
W is absent or is independently selected from O, S, NH, and N—C₁₋₆alkyl, preferably O;
Y is absent or is selected from O, NH, N—C₁₋₆alkyl, S, SO, SO₂, CHOR₁₀, and CHCO₂R¹⁰;
each Z is independently selected from O, S, NH, and N—C₁₋₆alkyl, preferably O; or
Z is optionally a covalent bond when adjacent to an occurrence of A;
R¹, R², R³, and R⁴ are each independently selected from C₁₋₆alkyl, C₁₋₆hydroxyalkyl, C₁₋₆alkoxyalkyl, aryl, C₁₋₆aralkyl, and R¹⁴DVKOC₁₋₃alkyl-, wherein at least one of R¹ and R³ is R¹⁴DVKOC₁₋₃alkyl-;
R⁵ is N(R⁶)LQR⁷;
R⁶ is selected from hydrogen, OH, and C₁₋₆alkyl, preferably C₁₋₆alkyl;
R⁷ is a further chain of amino acids, hydrogen, a protecting group, aryl, or heteroaryl, any of which is optionally substituted with halogen, carbonyl, nitro, hydroxy, aryl, C₁₋₅alkyl; or R⁷ is selected from C₁₋₆alkyl, C₁₋₆alkenyl, C₁₋₆alkynyl, C₁₋₆aralkyl, C₁₋₆heteroaralkyl, R⁸ZAZ-C₁₋₈alkyl-, R¹¹Z-C₁₋₈alkyl-, (R⁸O)(R⁹O)P(═O)O—C₁₋₈alkyl-ZAZ-C₁₋₈alkyl-, R⁸ZAZ-C₁₋₈alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH, wherein each occurrence of Z and A is independently other than a covalent bond; or
R⁶ and R⁷ together are C₁₋₆alkyl-Y—C₁₋₆alkyl, ZAZ-C₁₋₆alkyl-ZAZ-C₁₋₆alkyl, or ZAZ-C₁₋₆alkyl-ZAZ, thereby forming a ring, wherein each occurrence of Z and A is independently other than a covalent bond;
R⁵ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;
each R¹⁰ is independently selected from hydrogen and C₁₋₆alkyl, preferably C₁₋₆alkyl; each R¹¹ is independently selected from hydrogen, OR¹⁰, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl;
R¹⁴ is selected from hydrogen, (R¹⁵O)(R¹⁶O)P(═O)W—, R¹⁵GB-, heterocyclyl-, (R¹⁷)₂N—, (R¹⁷)₃N⁺—, R¹⁷SO₂GBG-, and R¹⁵GBC₁₋₈alkyl- where the C₁₋₈alkyl moiety is optionally substituted with OH, C₁₋₈alkylW (optionally substituted with halogen, preferably fluorine), aryl, heteroaryl, carbocyclyl, heterocyclyl, and C₁₋₆aralkyl, preferably at least one occurrence of R¹⁴ is other than hydrogen;
R¹⁵ and R¹⁶ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R¹⁵ and R¹⁶ together are C₁₋₆alkyl, thereby forming a ring; and
each R¹⁷ is independently selected from hydrogen, OR¹⁰, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl; provided that when R⁶ is H, L is C═O, and Q is absent, R⁷ is not hydrogen, C₁₋₆alkyl, or substituted or unsubstituted aryl or heteroaryl; and
D, G, V, K, and W are selected such that there are no O—O, N—O, S—N, or S—O bonds; and

In certain embodiments, the chain of amino acids has a structure of formula (V) or (VI) or a pharmaceutically acceptable salt thereof 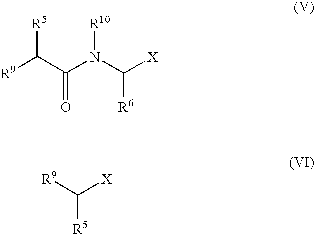
wherein
each Ar is independently an aromatic or heteroaromatic group optionally substituted with 1 to 4 substituents;
L is absent or is selected from C═O, C═S, and SO₂, preferably SO₂ or C═O;
X is COOH or an activated form thereof, preferably X is COOH, COCl, or CON(Me)(OMe), most preferably X is COOH or COCl;
Y is absent or is selected from C═O and SO₂;
Z is absent or is C₁₋₆alkyl;
R⁵ and R⁶ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₆alkyl ester, C₁₋₅alkyl ester, and aryl ester), thiol, or thioether substituents;
R⁹ is N(R¹⁰)L-Z-R¹¹;
R¹⁰ is selected from hydrogen, OH, C₁₋₆aralkyl-Y—, and C₁₋₆alkyl-Y—, preferably hydrogen;
R¹¹ is selected from hydrogen, OR¹², C₁₋₆alkenyl, Ar—Y—, carbocyclyl, and heterocyclyl; and
R¹² is selected from hydrogen, C₁₋₆alkyl, and C₁₋₆aralkyl, preferably hydrogen.

In certain embodiments, L is selected from C═O, C═S, and SO₂, preferably SO₂ or C═O.

In certain embodiments, R¹⁰ is selected from hydrogen, OH, C₁₋₆aralkyl, and C₁₋₆alkyl, preferably hydrogen.

In certain embodiments, R¹¹ is selected from hydrogen, C₁₋₆alkenyl, Ar—Y—, carbocyclyl, and heterocyclyl.

In certain embodiments, R⁵ and R⁶ are each independently selected from C₁₋₆alkyl, C₁₋₆hydroxyalkyl, and C₁₋₆aralkyl. In preferred such embodiments, R⁵ is C₁₋₆alkyl and R⁶ is C₁₋₆aralkyl. In more preferred such embodiments, R⁵ is isobutyl and R⁶ is phenylmethyl.

In certain embodiments, R¹⁰ is hydrogen, L is C═O or SO₂, R¹¹ is Ar—Y—, and each Ar is independently selected from phenyl, indolyl, benzofuranyl, naphthyl, quinolinyl, quinolonyl, thienyl, pyridyl, pyrazyl, and the like. In certain such embodiments, Ar may be substituted with Ar-Q-, where Q is selected from a direct bond, —O—, and C₁₋₆alkyl. In certain other such embodiments where Z is C₁₋₆alkyl, Z may be substituted, preferably with Ar, e.g., phenyl.

In certain embodiments, R¹⁰ is hydrogen, Z is absent, L is C═O or SO₂, and R¹¹ is selected from Ar—Y and heterocyclyl. In certain preferred such embodiments, heterocyclyl is selected from chromonyl, chromanyl, morpholino, and piperidinyl. In certain other preferred such embodiments, Ar is selected from phenyl, indolyl, benzofuranyl, naphthyl, quinolinyl, quinolonyl, thienyl, pyridyl, pyrazyl, and the like.

In certain embodiments, R¹⁰ is hydrogen, L is C═O or SO₂, Z is absent, and R¹¹ is C₁₋₆alkenyl, where C₁₋₆alkenyl is a substituted vinyl group where the substituent is preferably an aryl or heteroaryl group, more preferably a phenyl group optionally substituted with one to four substituents.

In certain embodiments, R¹² is selected from hydrogen and C₁₋₆alkyl. In certain preferred such embodiments, R¹² is selected from hydrogen and methyl. In more preferred such embodiments, R¹² is hydrogen.

In certain preferred embodiments, the chain of amino acids has a structure of formula (VII)

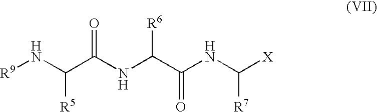

X is COOH or an activated form thereof, preferably X is COOH, COCl, or CON(Me)(OMe), most preferably X is COOH or COCl;

R⁵, R⁶, and R⁷ are independently selected from C₁₋₆alkyl, C₁₋₆hydroxyalkyl, C₁₋₆alkoxyalkyl, aryl, and C₁₋₆aralkyl, each of which is optionally substituted with a group selected from amide, amine, carboxylic acid or a pharmaceutically acceptable salt thereof, carboxyl ester, thiol, and thioether, preferably R⁶ is C₁₋₆alkyl and R⁵ and R⁷ are C₁₋₆aralkyl, most preferably, R⁶ is isobutyl, R⁵ is 2-phenylethyl, and R⁷ is phenylmethyl;

R⁹ is a further chain of amino acids, hydrogen, C₁₋₆acyl, a protecting group, aryl, or heteroaryl, where substituents include halogen, carbonyl, nitro, hydroxy, aryl, and C₁₋₅alkyl, preferably R⁹ is C₁₋₆acyl, most preferably R⁹ is acetyl.

The terms “amine” and “amino” are art-recognized and refer to both unsubstituted and substituted amines and salts thereof, e.g., a moiety that can be represented by the general formulae: 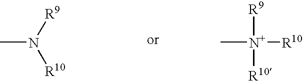
wherein R⁹, R¹⁰ and R¹⁰′ each independently represent a hydrogen, an alkyl, an alkenyl, —(CH₂)ₘ—R⁸, or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocyclyl or a polycyclyl; and m is zero or an integer from 1 to 8. In preferred embodiments, only one of R⁹ or R¹⁰ can be a carbonyl, e.g., R⁹, R¹⁰, and the nitrogen together do not form an imide. In even more preferred embodiments, R⁹ and R¹⁰ (and optionally R¹⁰′) each independently represent a hydrogen, an alkyl, an alkenyl, or —(CH₂)ₘ—R⁸. In certain embodiments, the amino group is basic, meaning the conjugate acid has a pKₐ>7.00.

The terms “amide” and “amido” are art-recognized as an amino-substituted carbonyl and includes a moiety that can be represented by the general formula: 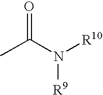
wherein R⁹ and R¹⁰ are as defined above for “amine” or “amino”. Preferred embodiments of the amide will not include imides which may be unstable.

The term “aryl” as used herein includes 5-, 6-, and 7-membered substituted or unsubstituted single-ring aromatic groups in which each atom of the ring is carbon. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The terms “carbocycle” and “carbocyclyl”, as used herein, refer to a non-aromatic substituted or unsubstituted ring in which each atom of the ring is carbon. The terms “carbocycle” and “carbocyclyl” also include polycyclic ring systems having two or more cyclic rings in which two or more carbons are common to two adjoining rings wherein at least one of the rings is carbocyclic, e.g., the other cyclic rings can be cycloalkyls, cycloalkenyls, cycloalkynyls, aryls, heteroaryls, and/or heterocyclyls.

The term “carbonyl” is art-recognized and includes such moieties as can be represented by the general formula: 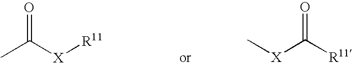
wherein X is a bond or represents an oxygen or a sulfur, and R¹¹ represents a hydrogen, an alkyl, an alkenyl, —(CH₂)ₘ—R⁸ or a pharmaceutically acceptable salt, R^{11′} represents a hydrogen, an alkyl, an alkenyl or —(CH₂)ₘ—R⁸, where m and R⁸ are as defined above. Where X is an oxygen and R¹¹ or R^{11′} is not hydrogen, the formula represents an “ester”. Where X is an oxygen, and R¹¹ is a hydrogen, the formula represents a “carboxylic acid”.

As used herein, “enzyme” can be any partially or wholly proteinaceous molecule which carries out a chemical reaction in a catalytic manner. Such enzymes can be native enzymes, fusion enzymes, proenzymes, apoenzymes, denatured enzymes, farnesylated enzymes, ubiquitinated enzymes, fatty acylated enzymes, gerangeranylated enzymes, GPI-linked enzymes, lipid-linked enzymes, prenylated enzymes, naturally-occurring or artificially-generated mutant enzymes, enzymes with side chain or backbone modifications, enzymes having leader sequences, and enzymes complexed with non-proteinaceous material, such as proteoglycans, proteoliposomes. Enzymes can be made by any means, including natural expression, promoted expression, cloning, various solution-based and solid-based peptide syntheses, and similar methods known to those of skill in the art.

The term “C₁₋₆heteroaralkyl”, as used herein, refers to a C₁₋₆alkyl group substituted with a heteroaryl group.

The terms “heteroaryl” includes substituted or unsubstituted aromatic 5- to 7-membered ring structures, more preferably 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, triazole, pyrazole, pyridine, pyrazine, pyridazine and pyrimidine, and the like.

The term “heteroatom” as used herein means an atom of any element other than carbon or hydrogen. Preferred heteroatoms are nitrogen, oxygen, phosphorus, and sulfur.

The terms “heterocyclyl” or “heterocyclic group” refer to substituted or unsubstituted non-aromatic 3- to 10-membered ring structures, more preferably 3- to 7-membered rings, whose ring structures include one to four heteroatoms. The term terms “heterocyclyl” or “heterocyclic group” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “C₁₋₆hydroxyalkyl” refers to a C₁₋₆alkyl group-substituted with a hydroxy group.

As used herein, the term “inhibitor” is meant to describe a compound that blocks or reduces an activity of an enzyme (for example, inhibition of proteolytic cleavage of standard fluorogenic peptide substrates such as Suc-LLVY-AMC, Box-LLR-AMC and Z-LLE-AMC, inhibition of various catalytic activities of the 20S proteasome). An inhibitor can act with competitive, uncompetitive, or noncompetitive inhibition. An inhibitor can bind reversibly or irreversibly, and therefore the term includes compounds that are suicide substrates of an enzyme. An inhibitor can modify one or more sites on or near the active site of the enzyme, or it can cause a conformational change elsewhere on the enzyme.

As used herein, the term “peptide” includes not only standard amide linkage with standard α-substituents, but commonly utilized peptidomimetics, other modified linkages, non-naturally occurring side chains, and side chain modifications, as detailed below.

The terms “polycyclyl” or “polycyclic” refer to two or more rings (e.g., cycloalkyls, cycloalkenyls, cycloalkynyls, aryls, heteroaryls, and/or heterocyclyls) in which two or more carbons are common to two adjoining rings, e.g., the rings are “fused rings”. Each of the rings of the polycycle can be substituted or unsubstituted.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “stereoselective” is art-recognized and refers to reactions in which one diastereomer (or one enantiomeric pair of diastereomers) is formed or destroyed in considerable preference to others that might have been formed or destroyed.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

A “therapeutically effective amount” of a compound with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner to improve or stabilize a subject's condition.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, flimarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In other cases, the inhibitors useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).
Exemplification 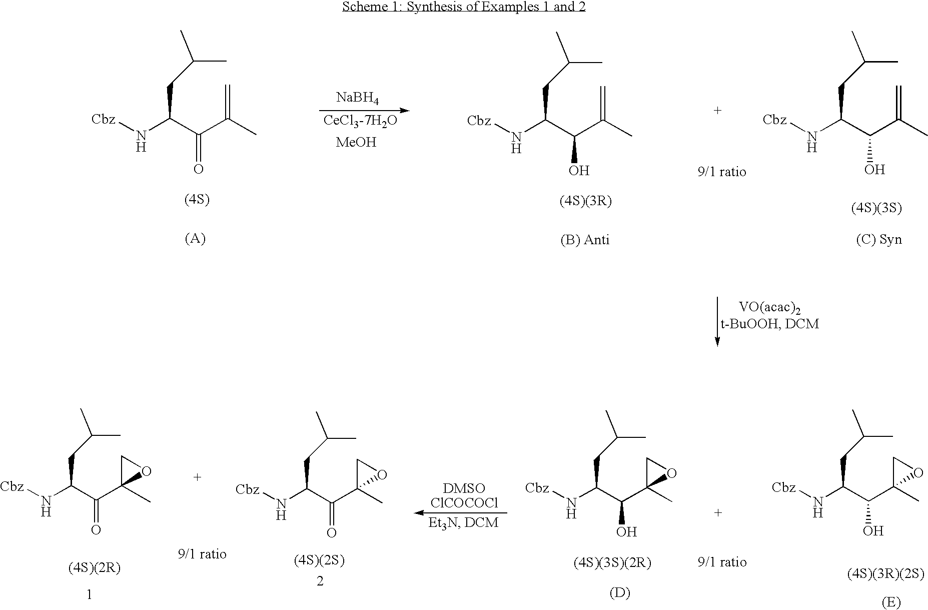
Synthesis of (B) and (C)

To a 0° C. solution of (A) (10.57 g, 36.52 mmol), [prepared as described in Bioorg. Med. Chem. Lett. 1999, 9, 2283-88] in 400 mL of MeOH was added CeCl₃-7H₂O (16.33 g, 43.82 mmol). The solution was stirred under an atmosphere of argon until the CeCl₃-7H₂O was completely dissolved. To this solution was added NaBH₄ (1.65 g, 43.82 mmol) in 10 portions over 2 minutes. The reaction was stirred under an atmosphere of argon at 0° C. for 6 hours, at which point the mixture became milky white. The reaction was quenched at 0° C. with approximately 5 mL of glacial HOAc and after 30 minutes of additional stirring at 0° C. the mixture became clear. The volatiles were removed under reduced pressure and the remaining oil taken up in EtOAc (300 mL). The organic layer was washed with water (2×200 mL), brine (2×200 mL) and dried over MgSO₄. The MgSO₄ was removed by filtration and the volatiles removed under reduced pressure. Purification by flash chromatography afforded (B) and (C) as a 9:1 mixture (10.5 g). R_{f}=0.21 (5:1 hexanes/EtOAc); ¹H NMR (300 MHz, CDCl₃): (B) δ 7.38-7.25 (m, 5 H), 5.18-4.82 (m, 5 H), 4.17 (br s, 1 H), 3.91 (m, 1 H), 1.99 (br s, 1 H), 1.78 (s, 3 H), 1.64 (m, 1 H), 1.26 (m, 2 H), 0.91 (m, 6 H).

Several reaction conditions were examined for the conversion of (A) to (B) and (C) as shown in table (1). In all cases the starting enone was completely consumed.

TABLE 1

| | | Temper- | Anti/Syn | |
|---|---|---|---|---|
| Reducing Agent | Solvent | ature | ratio | Comments |
| NaBH₄ | MeOH | 0° C. | 4:1 | |
| NaBH₄ | MeOH | 20° C. | 4:1 | |
| NaBH₄—CeCl₃—7H₂O | MeOH | 0° C. | 9:1 | |
| NaBH₄ | THF | 0° C. | 6:1 | Not as clean |
| | | | | as MeOH |
| L-Selectride | THF | −78° C. | 25:1 | 1,4-reduction |
| | | | | followed by |
| | | | | 1,2-reduction |
| L-Selectride- | THF | −78° C. | 25:1 | 1,4-reduction |
| CeCl₃—7H₂O | | | | followed by |
| | | | | 1,2-reduction |

Synthesis of (D) and (E)

To a 0° C. solution of a 9/1 mixture of (B) and (C) (10.56 g, 36.24 mmol) in 360 mL of dry dichloromethane under an atmosphere of argon was added VO(acac)₂ (0.289 g, 1.09 mmol). To this solution was added t-BuOOH (5.5 M solution in decane, 13.2 mL, 72.5 mmol). Upon the addition of the t-BuOOH the color of the reaction turned from a bright green to a deep red. The reaction was removed from the ice-water bath and allowed to warm to room temperature while stirring for 6 hours. At this time the color of the reaction had become light yellow and the reaction was deemed complete by TLC. The reaction mixture was filtered through a plug of Celite, transferred to a separatory funnel, and washed with ½ saturated NaHCO₃ (2×200 mL) and the aqueous layer extracted with dichloromethane (2×100 mL). The organic layers were combined and washed with water (2×200 mL) and brine (2×200 mL) and dried over MgSO₄. The MgSO₄ was removed by filtration and the volatiles removed under reduced pressure to give a 9/1 mixture of (D) and (E) (10.0 g). R_{f}=0.18 (3:1 hexanes/EtOAc); ¹H NMR (300 MHz, CDCl₃): (D) δ 7.38-7.31 (m, 5 H), 5.15-4.98 (m, 3 H), 3.99 (m, 1 H), 3.87 (br s, 1 H), 2.97 (d, J=4.69 Hz, 1 H), 2.64 (d, J=4.69 Hz, 1 H), 2.15 (br s, 1 H), 1.65 (m, 1 H), 1.47 (m, 1 H), 1.37 (s, 3 H), 1.09 (m, 1 H), 0.92 (m, 6 H). 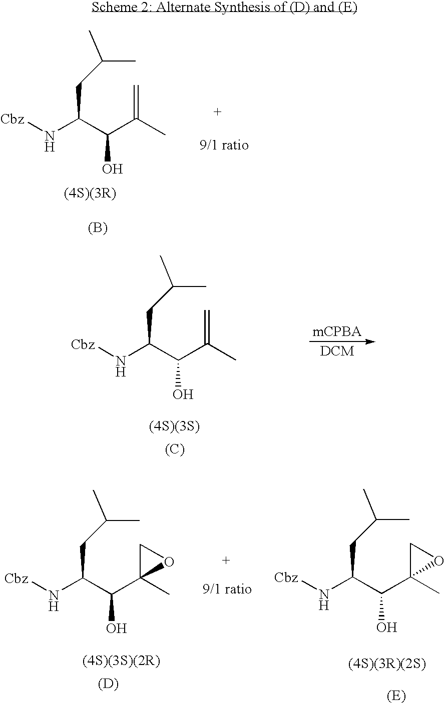
Alternate Synthesis of (D) and (E)

To a solution of a 9/1 mixture of (B) and (C) (0.34 mmol, 0.10 g) in DCM (5 mL) was added mCPBA (0.52 mmol, 0.089 g) and the mixture stirred for 5 hours. The mixture was diluted with sat. NaHCO₃ (5 mL) and the layers separated. The aqueous layer was extracted with DCM (2×5mL) and the layers combined, washed with sat. NaHCO₃ (3×15 mL), water (1×10 mL), brine (1×15 mL) and dried over MgSO₄. The MgSO₄ was removed by filtration and the volatiles removed under reduced pressure to give a 9/1 mixture of (D) and (E) (0.097 g).

Synthesis of Compounds 1 and 2

To 225 mL of dry dichloromethane was added oxalyl chloride (22.85 g, 15.7 mL, 180 mmol) and the mixture cooled to −78° C. under an atmosphere of argon. To the cooled solution was added drop wise, DMSO (16.88 g, 15.33 mL, 216 mmol), freshly distilled from CaH₂, dissolved in 60 mL of dry dichloromethane. The temperature of the reaction was monitored during the addition of the DMSO and the addition rate adjusted to keep the temperature below −65° C. requiring approximately 45 minutes. Ten minutes after the addition of the DMSO, a 9/1 mixture of (D) and (E) (10.0 g, 32.5 mmol) dissolved in 75 mL of dry dichloromethane was added drop wise. The reaction was stirred at −78° C. for 30 minutes and triethylamine (36.4 g, 50.6 mL, 360 mmol) was added drop wise and the reaction mixture placed in a 0° C. ice bath. The reaction was maintained at 0° C. for 30 minutes at which time the reaction was deemed complete by TLC. The reaction was quenched with 20 mL of H₂O mixed with 80 mL of dichloromethane. The muddy brown mixture became clear brown after approximately 10 minutes of stirring. The layers were separated and the aqueous layer extracted with CH₂Cl₂ (2×50 mL). The organic layers were combined washed with brine (2×100 mL) and dried over MgSO₄. The MgSO₄ was removed by filtration and the volatiles removed under reduced pressure. Purification by flash chromatography gave compound 1 (3.18 g). R_{f}=0.26 (5:1 hexanes/EtOAc); ¹H NMR (300 MHz, CDCl₃): δ 7.39-7.22 (m, 5 H), 5.11 (d, J=9.4 Hz, 1 H), 5.07 (d, J=15.5 Hz, 1 H), 5.03 (d, J=15.2 Hz, 1 H), 4.40 (ddd, J=10.3, 8.8, 2.9 Hz, 1 H), 3.27 (d, J=5.0, 1 H), 2.90 (d, J=5.0 Hz, 1 H), 1.71 (m, 1 H), 1.47 (s, 3 H), 1.21 (m, 2 H), 0.97 (d, J=6.5 Hz, 3 H), 0.93 (d, J=6.7 Hz, 3 H); ¹³C NMR (75 MHz, CDCl₃): δ 201.9, 156.2, 136.1, 128.5, 128.1, 128.0, 66.9, 52.2, 51.8, 40.5, 25.0, 23.4, 21.2, 16.7. Compound 2: R_{f}=0.15 (5:1 hexanes/EtOAc); ¹H NMR (300 MHz, CDCl₃): δ 7.39-7.25 (m, 5 H), 5.09 (m, 3 H), 4.66 (ddd, J=7.3, 7.0, 7.0 Hz, 1 H), 3.03 (d, J=4.7 Hz, 1 H), 2.86 (d, J=5.0 Hz, 1 H), 1.70 (m, 1 H), 1.55 (s, 3 H), 1.37 (m, 2 H), 0.97 (d, J=6.5 Hz, 3 H), 0.91 (d, J=6.7 Hz, 3 H); ¹³C NMR (75 MHz, CDCl₃): δ 207.4, 155.7, 136.1, 128.4, 128.1, 128.0, 66.9, 58.6, 53.0, 52.7, 41.0, 24.7, 23.2, 21.4, 17.4. 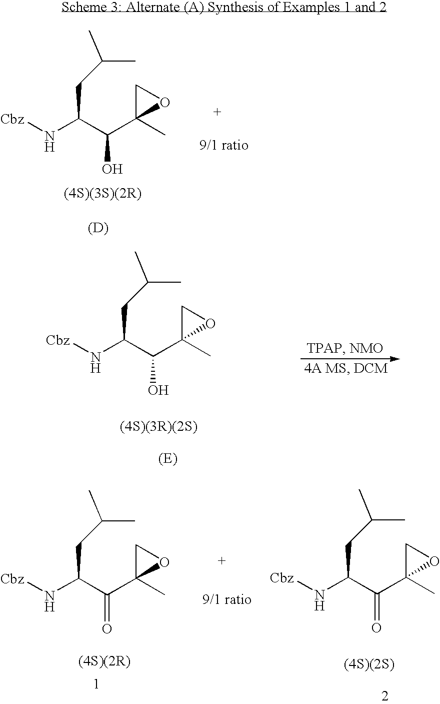
Alternate (A) Synthesis of 1 and 2

To a solution of a 9/1 mixture of (D) and (E) (0.29 g, 0.95 mmol) in DCM (10 mL) was added NMO (0.17 g, 1.43 mmol), crushed 4Å molecular sieves (0.76 g) and the reaction was stirred for 15 minutes. TPAP (0.02 g, 0.05 mmol) was added to the mixture and the reaction was stirred for 36 hours. The reaction was diluted with hexanes (15 mL) and filtered through a plug of silica gel, washing with (5:1 hexanes/EtOAc, 50 mL). The volatiles were removed under reduced pressure and the crude material purified by flash chromatography affording 1 (0.138 g). 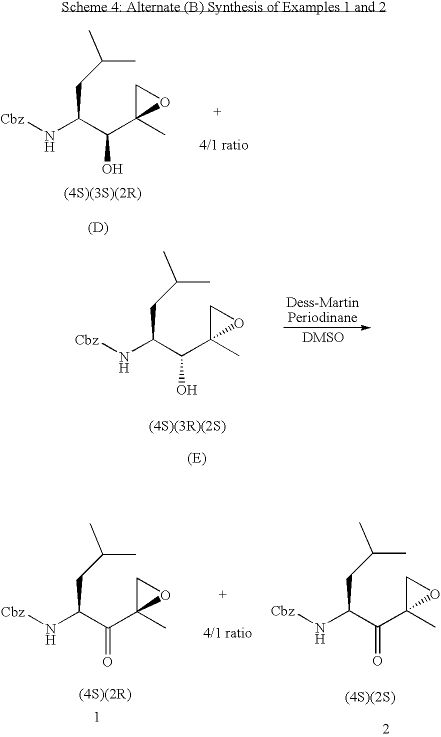
Alternate (B) Synthesis of 1 and 2

To a 5° C. solution of Dess-Martin Periodinane (6.95 g, 16.4 mmol) in 80 mL DMSO was added a 4/1 mixture of (D) and (E) (2.52 g, 8.20 mmol) as a DMSO solution (15 mL). The mixture was placed under an atmosphere of argon and allowed to warm to room temperature while stirring overnight. When complete, a white precipitate had formed and the reaction was cooled in an ice-bath and diluted with 100 mL sat. NaHCO₃. The mixture was further diluted with 400 mL of EtOAc and the solids removed by filtering through a plug of Celite. The mixture was transferred to a separatory funnel and the layers separated. The aqueous layer was extracted with EtOAc (2×200 mL) and the organic layers combined, washed with H₂O (3×100 mL), brine (1×400 mL) and dried over Na₂SO₄. The Na₂SO₄ was removed by filtration and the volatiles removed under reduced pressure to give a light yellow oil. Purification by flash chromatography afforded 1 (1.0 g) and 2 (0.20 g). 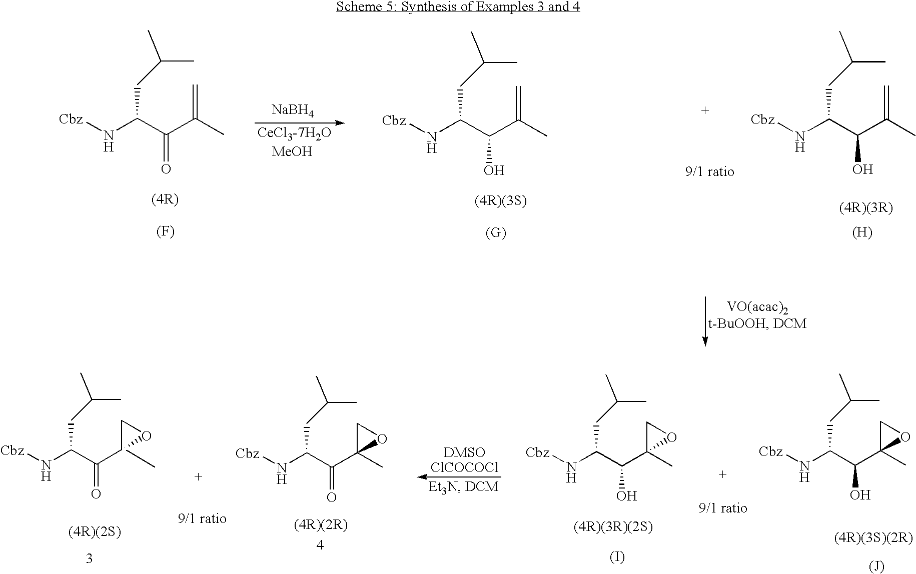
Synthesis of (G) and (H)

Compounds (G) and (H) were obtained by following the same procedure for the conversion of (A) to (B) and (C) but substituting (F) for (A).

Synthesis of (I) and (J)

Compounds (I) and (J) were obtained by following the same procedure for the conversion of (B) and (C) to (D) and (E) but substituting (G) and (H) for (B) and (C) respectively.

Synthesis of Compounds 3 and 4

Compounds 3 and 4 were obtained by following the same procedure for the conversion of (D) and (E) to compounds 1 and 2 respectively but substituting (I) and (J) for (D) and (E) respectively. The ¹H and ¹³C NMR spectra of compounds 3 and 4 were identical to compounds 1 and 2 respectively.

Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims.

All of the above-cited references and publications are hereby incorporated by reference.

1. A method for the synthesis of amino acid keto-epoxides comprising a sequence of reactions according to scheme (I) 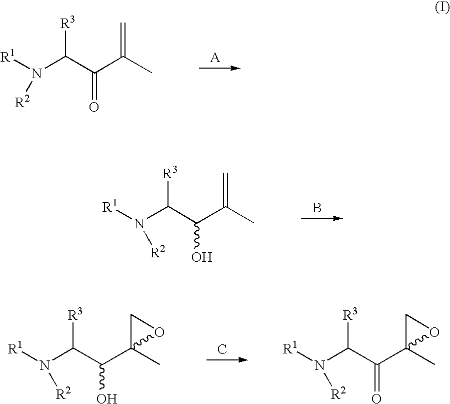
wherein
R¹ is selected from a protecting group or a further chain of amino acids, which itself may be optionally substituted;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, aryl, and C₁₋₆aralkyl;
A is a stereoselective reduction under reducing conditions;
B is a stereoselective epoxidation under epoxidizing conditions; and
C is an oxidation under oxidizing conditions:

2. A method of claim 1, wherein R¹ is a protecting group.

3. A method of claim 2, wherein R¹ is an electron withdrawing protecting group.

4. A method of claim 3, wherein R¹ is selected from t-butoxy carbonyl (Boc), benzoyl (Bz), fluoren-9-ylmethoxycarbonyl (Fmoc), trichloroethoxycarbonyl (Troc), and benzyloxy carbonyl (Cbz).

5. A method of claim 4, wherein R¹ is Cbz.

6. A method of claim 4, wherein R² is hydrogen.

7. A method of claim 1, wherein A is selected from sodium borohydride with cerium trichloride, lithium tri-tert-butoxyaluminum hydride, or L-selectride.

8. A method of claim 7, wherein A is sodium borohydride with cerium trichloride.

9. A method of claim 1, wherein B is selected from m-chloroperbenzoic acid or VO(acac)₂ with t-BuOOH.

10. A method of claim 9, wherein B is VO(acac)₂ with t-BuOOH.

11. A method of claim 1, wherein C is selected from a Swern oxidation or an oxidation wherein the oxidizing reagent(s) is Dess-Martin periodinane or tetrapropylammonium perruthenate (TPAP) with 4-methylmorpholine-N-oxide (NMO).

12. A method of claim 11, wherein C is a Swern oxidation.

13. A method of claim 11, wherein C is a oxidation with Dess-Martin periodinane.

14. A method of claim 1, wherein the compounds in scheme (I) have the following stereochemistry

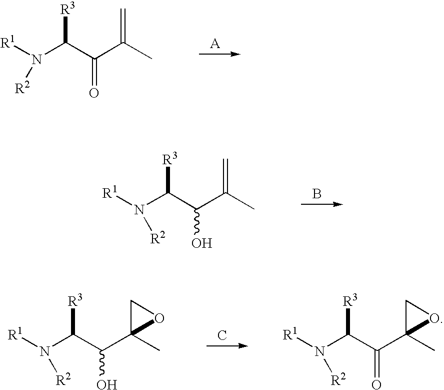

15. A method of claim 1, further comprising removing the protecting group if necessary and coupling with a chain of amino acids.

16. A method of claim 15, wherein the chain of amino acids comprises three amino acids.

17. A method of claim 16, wherein the chain of amino acids has a structure of formula (VII) 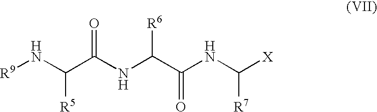
is X is COOH or an activated form thereof;
R⁵, R⁶, and R⁷ are independently selected from C₁₋₆alkyl, C₁₋₆hydroxyalkyl, C₁₋₆alkoxyalkyl, aryl, and C₁₋₆aralkyl, each of which is optionally substituted with a group selected from amide, amine, carboxylic acid or a pharmaceutically acceptable salt thereof, carboxyl ester, thiol, and thioether;
R⁹ is a further chain of amino acids, hydrogen, C₁₋₆acyl, a protecting group, aryl, or heteroaryl, where substituents include halogen, carbonyl, nitro, hydroxy, aryl, and C₁₋₅alkyl.

18. A method of claim 17, wherein X is COOH or COCl, R⁵ and R⁷ are C₁₋₆aralkyl, R⁶ is C₁₋₆alkyl, and R⁹ is C₁₋₆acyl.

19. A method of claim 18, wherein X is COOH, R⁵ is 2-phenylethyl, R⁶ is isobutyl, R⁷ is phenylmethyl, and R⁹ is acetyl.

20. A method for the synthesis of an allyl alcohol according to scheme (II) 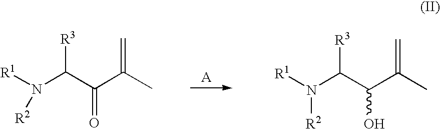
wherein
R¹ is selected from a protecting group or a further chain of amino acids, which itself may be optionally substituted;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, aryl, and C₁₋₆aralkyl; and
A is a stereoselective reduction under reducing conditions.

21. A method for the synthesis of an epoxide according to scheme (III) 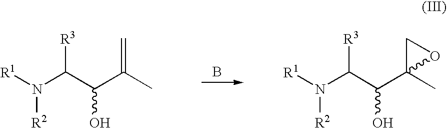
wherein
R¹ is selected from a protecting group or a further chain of amino acids, which itself may be optionally substituted;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, aryl, and C₁₋₆aralkyl; and
B is a stereoselective epoxidation under epoxidizing conditions.

22. A method for the synthesis of amino acid keto-epoxides according to scheme (IV) 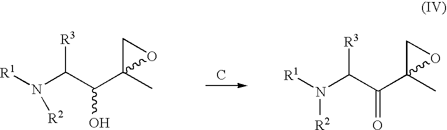
wherein
R¹ is selected from a protecting group or a further chain of amino acids, which itself may be optionally substituted;
R² is selected from hydrogen and C₁₋₆alkyl; or
R¹ and R² together are C(O)-aryl-C(O) or C(O)C₁₋₆alkenylC(O), thereby forming a ring;
R³ is selected from hydrogen, C₁₋₆alkyl, C₁₋₆alkoxyalkyl, aryl, and C₁₋₆aralkyl; and
C is an oxidation under oxidizing conditions.
